(19) United States
(12) Patent Application Publication
(10) Pub. No.: US 2008/0172936 A1
(43) Pub. Date: Jul. 24, 2008

(54) Method of collecting seeds

(75) Inventors: Andreas Boon, Totiusdal (ZA); Ralph George Peckover, Pyramid (ZA)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc., d/b/a UNILEVER

(21) Appl. No.: 12/009,285

(22) Filed: Jan. 17, 2008

Related U.S. Application Data
(60) Provisional application No. 60/966,561, filed on Jan. 22, 2007.

(51) Int. Cl. A01H 5/10; A01H 5/00

(52) U.S. Cl. 47/58.1 R; 800/298

(57) ABSTRACT

A method of collecting seeds from a plant of Apocynaceae family, using a special implement (tubulet) for the seedpods, ripe seedpods covered with the tubulets, and seeds obtained by the method.

This application claims the benefit of U.S. provisional application No. 60/966,561 filed on Jan. 22, 2007.

### FIELD OF THE INVENTION The present invention relates to collecting seeds and seedpods from plants of Apocynaceae family.

### BACKGROUND OF THE INVENTION

Apocynaceae family plants are succulent desert family of plants, which includes Hoodia, Caralluma, Orbea, Stapelia, and Lavrania genera. Certain actives obtainable from Hoodia plants, e.g. steroidal glycosides, have been shown to have appetite suppressant activity and to be useful in weight management products. Many of these species, e.g. Hoodia, are on the endangered list, so that collection of the wild plants is not possible. Commercial cultivation and harvesting of Apocynacea plants has become of interest.

Unfortunately, the cultivation of many species is fraught with difficulties. A crucial stage of cultivation is collecting an abundant harvest of healthy seeds for future plantings. At maturity the seedpod gradually splits open and the seeds, conveniently equipped with little parachutes (feathery appendage located at one end of the seed), gradually exit the seedpod by e.g. wind movement and are carried by air currents in various directions and to various distances from the parent plant. Thus, much of the seed is lost. The remaining fraction has to be collected from the ground, which may impair the quality of the seed.

The use of female nylon stocking, netting, mosquito mesh, and cheese cloth on plants other than those of Apocynacea family has been described. See, for instance, Collecting Seeds, Burke's Backyard, 1999; Fort Irwin—Seed Collection, Soil Ecology Restoration Group, 9 Apr. 9 1998; Fidelibus et al., Native Seed collection, processing, and storage for revegetation projects, Restoration in the Colorado Desert: Management Notes, October 1993.

Unfortunately, the various materials described in these documents, do not provide commercially acceptable solution to the unique seed preservation challenges posed by the Apocynacea plants. The seedpods, which look like stalks, are of variable dimensions—some are thin and some are fatter; the dimensions also change as the seedpods grow, and then dry out. Furthermore, the seedpods grow up in a diagonal direction. The previously known protective materials do not fit snugly enough: they either fall off or fit too tightly if tied around the seedpod (e.g., female stocking tied around the seedpod) which damages the seedpod and does not allow for growth. Any commercially acceptable implement also needs to be re-useable—another challenging requirement, as the Apocynacea plants grow under harsh desert conditions, and the seedpods develop over a relatively long period.

### SUMMARY OF THE INVENTION

The present invention includes a method of collecting seeds from a plant of Apocynaceae family, the method comprising:
(a) placing a tubulet over a well-developed seedpod, wherein the tubulet is elastic along the transversal axis and has an opening on both ends of the longitudinal axis and is made of a woven or knit textile having a thickness of at least about 0.1 mm;
(b) allowing the seedpod to ripen within the tubulet;
(c) separating the ripe seedpod from the plant;
(d) removing the ripe seedpods from the tubulet,
(e) collecting the seed.

The invention further includes a ripe seedpod, separated from an Apocynaceae plant, the seedpod covered with a tubulet which is elastic along the transversal axis and has an opening on both ends of the longitudinal axis and is made of a woven or knit textile having a thickness of at least about 0.1 mm.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.”

It should be noted that in specifying any range, any particular upper limit can be associated with any particular lower limit.

For the avoidance of doubt the word “comprising” is intended to mean “including” but not necessarily “consisting of” or “composed of.” In other words, the listed steps or options need not be exhaustive.

### Apocynacea Plants

The invention is useful for collecting the seeds of plants of Apocynacea family, which includes but is not limited to Hoodia, Caralluma, Orbea, Stapelia, and Lavrania genera, more preferably the Hoodia plants, as the demand for appetite suppressant actives derived from the Hoodia plants continues to grow. The Hoodia genus includes but is not limited to Hoodia gordonii, Hoodia currorii, Hoodia juttae, Hoodia dregei, Hoodia parviflora, Hoodia pilifera, Hoodia alstonii, Hoodia flava, Hoodia officinalis, Hoodia mossamedensis, Hoodia triebneri, Hoodia pedicellata, Hoodia rushii. The invention is particularly valuable for Hoodia gordonii plants, as the actives derived from Hoodia gordonii plants have optimum safety and efficacy for weight management products.

### Elastic Tubulet

The elastic tubulet suitable for the present invention is a small tube-like textile implement that has an opening at both ends of the longitudinal axis (as in the tube) and is made of an elastic woven or knit textile. The particular virtue of the inventive tubulet is its precise fit to the seedpod, due to its elasticity and its dimensions.

The elasticity at least along the transversal axis is critical: the inventive tubulet is not merely stretchable but is elastic, i.e. it is capable of returning to the initial shape after deformation; it is resilient to stretching. An implement that is not stretchable at all or is too stretchable without being elastic is difficult to apply and/or falls off. The tubulet is elastic in transversal direction and preferably also elastic in the longtitudinal direction: in the absence of a two-way stretch, the tubulets are not as easy to apply and don't fit as well.

Preferably, the textile has an elasticity of at least 30%, more preferably 30-200%, even more preferably 40-150%, and most preferably 50-100%, in both longitudinal direction and transversal direction, under forces excercised by a ripening seedpod of the Apocynaceae family, wherein an elasticity of X % is to be understood as an increase in length by X % under the forces referred to. Preferably, the elasticity is for at least 90% reversible, which is to be understood that if the forces that stretched it are withdrawn, the textile returns to its original shape to such an extent that at least 90% of the increase in length is reversed. This allows the textile to stay on, even after the seedpod reduces in size again. The ability to revert to such great extent to its original shape and size also allows the tubulet to be better re-used in future seed-collecting.

According to the present invention, after being placed on the seedpod, the elastic nature of the tubulet allows it to hug the seedpod snugly (the tubulet “grabs” the seedpod, regardless of whether the particular seedpod is relatively thin or fat. Thereafter, the tubulet continues to stretch with the growth of the seedpod, allowing the seedpod to grow and mature unimpeded, while also fitting closely enough to substantially minimize (in combination with pesticides) the access to the seedpod by tiny pests. When the seedpod finally ripens and starts to dry, the tubulet continues to follow the shape of the seedpod and continues to fit snugly, instead of falling off.

The durability and reusability of the tubulets is enhanced according to the present invention by the thickness of the textile from which the tubulet is made, in addition to being elastic. The textile according to the invention has thickness of at least 0.1 mm, preferably at least 0.2 mm, most preferably at least 0.5 mm, generally in the range from 0.1 to 1 mm, preferably from 0.2 to 0.8 mm, most preferably from 0.5 to 0.8 mm (compare to the typical female nylon stocking thickness of 0.05 mm or below or nylon tights—about 0.49 mm, but not sufficiently stretchable or elastic). It is a particular advantage of the inventive tubulets, that despite being quite thick to optimize re-usability, the material of the tubulets is yet air-permeable and allows for light transmission, which is of course important for the optimum health and development of the seedpod. A fabric with good air permeability is essential in order for the seedpod to continue its normal gas (CO2 and O2) exchange function.

The particular dimensions of the tubulet depend on the particular plant species, but must be such as to preferably cover at least about 60% of the seedpod, preferably at least 75% of the seedpod lengthwise, and also to fit snugly widthwise. For instance, the seedpods of Hoodia gordonii plants have the following average dimensions:

generally, Length: 100-200 mm, width :7.5-16 mm;

preferably, length: 150 mm; width: 12 mm-15 mm.

The tubulet should fit both thick and thin seedpods, preferably it should cover at Least 60% of the length of the seedpod, most preferably at least 75% of the seedpod.

When the inventive tubulets are intended for Hoodia plants, especially Hoodia gordonii plants, the dimensions of the tubulet are from 100 mm to 200 mm, preferably from 130 mm to 160 mm long and from 15 mm to 23 mm wide (depending on type of fabric and elasticity of the fabric), optimally 150 mm long and 20 mm wide (these dimensions including the stitch). The circumference of a seed pod would vary from 30-50 mm and should be able to be covered with a tubulet by means of stretching the fabric.

The reusability of tubulets is of the utmost importance in commercial cultivation, where millions of tubulets may be required for one round of harvest. The preferred tubulets are also resistant to tears or “runs.” Furthermore, the tubulet should preferably have UV resistance, since it is exposed to harsh UV radiation for quite Long periods of time. Thus, the preferred tubulets have UV resistance coating/finish and/or are made of textiles that are more UV resistant. The preferred tubulets are white or light colored, e.g. beige, in order to minimize heat from the sun exposure.

To optimize the durability of the tubulets, they are preferably manufactured without seams (although this is costly) or are produced with the material doubled over, so that there is only a single seam along one side of the tubulet along the longitudinal axis. The seam is a point of weakness so the fewer seams, the stronger the implement. The seam is preferably sown with a double stitch and further with a safety stitch to ensure the optimum strength of the seam.

Suitable materials from which textiles for the tubulets in the present invention are made include fibers based on synthetic organic polymers, including but not limited to polyester, polyamide, acrylic, polyurethane, and mixtures thereof. Preferably, the fiber bundles of the textile material are ordered and knit together in a neat, regular fabric. Preferably the fabric is substantially free of cotton (excluding stitch thread), generally comprising less than 10% cotton, preferably less than 5% cotton, most preferably less than 1% cotton, optimally 0%. The absence of the fibre bundles and/or more random the structure of the fabric may lead to undesirable tears or “runs” in the fabric.

### Method of Using

The inventive method is for collecting the seeds from Apocynacea plants while also protecting the seedpods and seeds from predators.

The seedpods of Apocynaceae plants undergo several stages of development after pollination. In the first stage, the seedpod is developed—this is the period during which the seedpod develops from the young primordial stage into a developed seedpod. In the primordial stage, the seedpods are usually dark purple in color and are soft. In the second stage, the developed seedpod grows and develops further into a well-developed seedpod which is a lighter green/brown/purple in color and has a firm feel. In the third stage, the well-developed seedpod turns into a mature or ripe seedpod, displaying a green/grayish color. The key signs indicating the readiness of the mature or ripe seedpod for harvest/collection in the next few days include the seedpods completely drying out, changing color from gray to yellow, and showing signs of splitting. The time period between the first stage and the second stage is about one month. The time period between well-developed seedpod and the mature/ripe stage is also approximately one month.

According to the inventive method, the tubulet is placed over a well-developed seedpod (at the beginning of the second stage of development—once the seedpod colors from purple to a lighter green and the seedpod becomes more firm). The tubulet should not be placed on the seedpod in the developing stage, since that would hinder the normal development of the seedpod, e.g. it could deform the seedpod or slow down the growth of the seedpod. The elastic tubulet is able to allow the well-developed seedpod to grow and mature and then dry somewhat, while maintaining the snug fit over the seedpod, while also being air and light permeable, allowing the seedpod to breath and photosynthesize normally. Once the seedpod is sufficiently matured, it is collected. The collection may be done by hand or through mechanical means.

To minimize the exposure to the predators, pesticides may be applied. Pesticides are applied with the aim to target the seedpods and tubulets specifically. Pesticides can be applied manually, through use of a knapsack sprayer which is carried by a protected, trained and certified operator, or they can be applied by using a boom sprayer which is mounted on a tractor which is guided by a protected, trained and certified operator. Pesticides may also be applied on top of tubulets, preferably after the tubulets have been placed on the seedpods.

The tubulet can be applied to the seedpods throughout the seed production period whenever the seedpod is mature. Application of tubulets can be combined with pesticide application and seed collection actions.

According to the present invention, the mature seedpod is allowed to dry fully prior to collecting the seed. Pods will always split open when fully dry, actually they begin to split early in the morning when the seedpods are still wet and during the day dry out and gradually release the seed. Preferably the seedpods are collected before they split open, although some may have split open prior to collection. In the latter case, the tubulet also prevents the substantial majority of the seeds from flying away or dropping on the ground. In the preferred embodiment of the invention, the mature seedpod, still within the tubulet, is separated from the plant. This can be done in any known fashion, preferably by cutting, using a knife or scissors (or just plucked), so that the seedpods are cleanly cut away from the parent plant preventing unnecessary damage. It is important to harvest rather quite dried out seed pods than seed pods with some moisture—this wilt eliminate fungal infections during the storage time before seed cleaning. The separated ripe seedpods with tubulets still enclosing them are then collected and air dried. Any known drying method may be used, preferably air drying (the most economical). Preferably seedpods with tubulets are placed (lightly packed to improve airflow) into air permeable, loose-weave (e.g., nylon) bags and are hung in a dry room with sufficient air flow (may use fan). This is to complete the total drying out of the seedpods and the associated seeds. It will minimize the risk that the seedpods and the seeds will rot due to the remaining moisture in storage. The seeds pods are then liberated from the tubulets, which may be aided by pushing the contents of the tubulet out with a rod, and the seeds are separated and collected.

The following specific examples further illustrate the invention, but the invention is not limited thereto.

### Examples 1-5

### Comparative Examples A-B

The examples investigated the effect of tubulets made of various textiles and constructions on Hoodia gordonii seedpods. Tubelets in Examples 1-5 (all within the scope of the invention) were elastic; were about 150 mm long and about 15 mm wide (excluding stitch) and had single seam along longitudinal axis.

The results and observations that were collected are summarised in Table 1.

TABLE 1

| | | Textile | | |
|---|---|---|---|---|
| | Textile | Thickness | Seam | |
| Example | Material | (mm) | Construction | Observations |
| 1 (CVV) | Polyester; circular | Average: | Single stitch | Acceptable |
| | knitted fabric; | 0.529 mm | | |
| | 130 g/m² | st dev: | | |
| | | 0.007 | | |
| 2 (Rawsilk) | Acrylic | Average: | Double stitch | Acceptable, but |
| | (tripolene) | 0.63 mm | | produced the most |
| | | st dev: | | “runs” in the |
| | | 0.012 | | tubulet |
| 3 (dela-plast) | Texturised | Average: | Double stitch | Acceptable |
| | polyester; tribal | 0.529 mm | plus safety | |
| | interlock; wales | st dev: | stitch. | |
| | (rectangular to | 0.007 | Stitch length: | |
| | the course) 18 per cm; | | 2.55 mm | |
| | 130 g/m² | | | |
| 4 (sky-1) | Segmented | Average: | Single stitch | Very good |
| | polyurethane | 0.55 mm | | elasticity, more |
| | (Lycra) | st dev: | | expensive. |
| | | 0.008 | | |
| 5 (sky-2) | Polyester | Average: | Single stitch | Sometimes the |
| | (trilobal, | 0.606 mm | | width of these |
| | polyester) | st dev: | | tubulets was too |
| | | 0.005 | | small resulting in a |
| | | | | bad or no fit over |
| | | | | seed pod (not |
| | | | | optimally elastic). |
| A: Nylon | 100% Nylon | Average: | N/A | Very tight, not |
| tights | (knitted) | 0.489 mm | | sufficiently |
| | | st dev: | | stretchable or |
| | | 0.005 | | elastic. |
| B: Nylon | 100% Nylon | Average: | N/A | Very stretchable, |
| stocking | | 0.0035 mm | | runs resut easily. |

The following additional field observations have been made which illustrate [earnings in relation to the other types of tubulets and/or methods of applying tubulets tested:
1) application of a tubulet which is made from pure cotton and/or a includes cotton in the material mix has shown to cause degradation of the tubulets due to solar radiation and absorbing moisture during rainfall events—no elasticity with cotton.
2) application of a tubulet which is made from pure cotton and/or a includes cotton in the material mix has shown to be inappropriate as it does not stretch with the developing seedpods. The tubulet was therefore either too tight or too loose to secure a snug fit around the seedpod.
3) application of a tubulet which is made from pure cotton and/or a includes cotton in the material mix has shown to increase the incidence of seedpods being attacked by pests and diseases. This was due to either the tubulet itself degrading (as described under 1) or the tubulet not securing a snug fit (as described under 2). Both scenarios allowed easy entry of pests and diseases.
4) application of a tubulet covering less than 60% of the length of the seedpod has shown insufficient protection of the seedpod from pests and diseases.
5) application of a tubulet covering less than 60% of the length of the seedpod has shown insufficient protection of the seedpod from loosing seed from the pod during the final stages of maturation.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable.

What is claimed is:

1. A method of collecting seeds from a plant of Apocynaceae family, the method comprising:
(a) placing a tubulet over a well-developed seedpod, wherein the tubulet is elastic along the transversal axis and has an opening on both ends of the longitudinal axis and is made of a woven or knit textile having a thickness of at least about 0.1 mm;
(b) allowing the seedpod to ripen within the tubulet;
(c) separating the ripe seedpod from the plant;
(d) removing the ripe seedpods from the tubulet,
(e) collecting the seed.

2. The method of claim 1 wherein the tubulet covers at least about 60% of the length of seedpod.

3. The method of claim 1 wherein the tubulet covers at least about 75% of the seedpod.

4. The method of claim 1 wherein the tubulet dimensions are from about 100 mm to about 200 mm long and from about 13 mm to about 20 mm wide when flat, excluding stitch.

5. The method of claim 1 wherein the tubulet is elastic in both transversal and longitudinal directions.

6. The method of claim 5 wherein the textile has an elasticity of at Least 30%, preferably 30-200%, more preferably 40-150%, most preferably 50-100%, in both longitudinal direction and transversal direction, under forces excercised by a ripening seedpod of the Apocynaceae family.

7. The method of claim 1 wherein the textile has the thickness of at least 0.2 mm.

8. The method of claim 1 wherein the textile has the thickness of at least 0.5 mm.

9. The method of claim 1 wherein the tubulet is re-useable.

10. The method of claim 1 wherein the tubulet is resistant to tear.

11. The method of claim 1 wherein the tubulet comprises a single seam along the longtitudinal axis.

12. The method of claim 11 wherein the seam is made with a double stitch.

13. The method of claim 11 wherein the seam further comprises a safety stitch.

14. The method of claim 11 wherein the seam is made with a single stitch.

15. The method of claim 1 wherein the textile is air permeable.

16. The method of claim 1 wherein the textile comprises fiber based on a synthetic organic polymer.

17. The method of claim 16 wherein the fiber is selected from polyester, polyamide, acrylic, polyurethane, and mixtures thereof.

18. The method of claim 1 wherein the tubulet is white or light-colored.

19. The method of claim 1 wherein the seedpod is separated from the plant by means of cutting or plucking.

20. The method of claim 1 further comprising drying the separated ripe seedpod within the tubulet.

21. The method of claim 1 wherein the plant is selected from the plants of Hoodia genus.

22. The method of claim 21 wherein the plant is Hoodia gordonii.

23. A ripe seedpod, separated from an Apocynaceae plant, the seedpod covered with a tubulet which is elastic along the transversal axis and has an opening on both ends of the longitudinal axis and is made of a woven or knit textile having a thickness of at least about 0.1 mm.
